(19) United States
(12) Patent Application Publication
(10) Pub. No.: US 2007/0235042 A1
(43) Pub. Date: Oct. 11, 2007

(54) Patients turn a kit

(76) Inventor: Sheila R. Vanderkam, Houston, TX (US)

Correspondence Address: Sheila R. Vanderkam, 406 McGowen, Houston, TX, 77006, US

(21) Appl. No.: 11/727,599

(22) Filed: Mar. 27, 2007

Related U.S. Application Data
(60) Provisional application No. 60/744,657, filed on Apr. 11, 2006.

(51) Int. Cl. A61B 19/00; A61F 5/37

(52) U.S. Cl. 128/876; 128/869

(57) ABSTRACT

A device that is used to turn a non-ambulatory patient in a hospital bed from left to right. The device has a padded cushion made with a single sheet of washable vinyl material and is filled with a poly-down bonded quilt batting which is placed inside of the vinyl sheet and covered with a cotton lining. This padded cushion is placed in the center of the device. There are two nylon straps that run through the length of the cushion and are encased in a sleeve that is sewn into the cotton lining which extends out of both ends if the padding to be placed on the top and bottom bed rails. At the end of both straps are Velcro and two metal grommets in which metal, plastic clamps, or restraint straps are used to affix the straps to the bed railing holding patient in position.

### BACKGROUND OF THE INVENTION (DESCRIPTION)

The device is an aid to personnel who attend patients that are in need of turning. The device applies the principal of leverage so that a single hospital worker can easily turn a comatose or non-ambulatory patient from one side of the bed to the other with safety to the patient and without injury to the worker. This device is placed underneath the waist area of the patient. Currently, most hospitals and or nursing facilities may need two more staff members to turn such persons, depending on the size and weight of the patient. The apparatus is comprised of the following components:
Industrial nylon strap/belts are used to turn the patient from left to right. Velcro is used to hold the center vinyl cushion in place (inside polyester fiberfill material) which is used to protect the patients abdominal and other areas of the waist area that is being supported. The Velcro is also used at both ends of the strap/belt for added support which is attached to the bed rails.
Metal holes (Grommets) at each end of the device will be secured with metal clips, hard plastic clamps, or a strong material straps (same as used for restraints) to hold a patient on their side.
Pull over rubber grips are used on the bed rails to keep the straps in place.

The apparatus is used to support and turn the patient from one side of the bed to the other. This helps to alleviate bedsores, assists in bathing, and provides keeping the patient moving while comatose, or non-ambulatory.

metal clamps, plastic clamps, or restraining straps.

### SUMMARY OF THE INVENTION

The invention is a strap/belt made of industrial nylon material with an adjustable padded center support consisting of polyester fiber filled material. Although the nylon material is washable (A disposable fitted pillow casing is used for sanitation purposes on the padding itself) it is also soft and comfortable which aids in the protection of the patient's skin. (Used to fit around the patient's waist area) This device will enable a single person to easily turn non-ambulatory patients. The end of each strap has two grommets which can be secured with either metal, hard plastic clamps or restraint straps to the bed railings.

The device uses the concept of leverage to enable a single or smaller person to easily turn non-ambulatory patients or to hold a patient in place for medical procedures or bathing.

### BRIEF DESCRIPTION OF THE DRAWINGS

FIG. 1 is a rear view of the inside position of the devices' padded nylon cushioned center which has side slits on both sides through which the industrial material strap/belt is threaded.

FIG. 2 is the strap/belt rear view of a strap/belt made of industrial material which is used to secure the apparatus when placed on the patient to the bed railing.

FIG. 3 is the rear view of the strap/belt Velcro material which is attached to each end of the strap/belt.

FIG. 4 is one of the two metal grommets inside view is used as access for a metal, hard plastic clamps, or strip of restraining straps and is placed through the Velcro at each end of the strap/belt.

FIG. 5 is perspective front view of the strap/belt that is made up of industrial material that is threaded through the padded center and used to secure the apparatus to the bed railings.

FIG. 6 is the frontal view of the outside of the devices' padded nylon cushion center which has side slits through which the strap/belt threaded.

FIG. 7 is a top view of the strap/belt with the grommets at each end which is used to hold the metal, plastic clamps, or restraint straps to the bed railings.

FIG. 8 is a top view of the strap/belt that has a small strip of Velcro in the center of the strap/belt used to hold the padded center in place.

FIG. 9 is an enlarge frontal view of the grommets in which the metal, plastic, or restraint straps are used.

FIG. 10 is an inside view (as if cut in half) of the center pad that is made of a thin soft medical polyester fiber filled material which is used to support and protect the patients.

FIG. 11 is an inside view (as if cut in half) are four strips of Velcro that is attached at each end of the padded area which is the entry point of the strap/belt.

FIG. 12 is a frontal view of the restraint strap is used as support in place of the metal or plastic clamps.

FIG. 13 is a side view of a metal or plastic clamp which is threaded through the grommets and used to secure the device to the bed railings.

FIG. 14 is a frontal view of the rubber grip used as extra protection to hold apparatus safely in place while attached to the bed rail(s).

FIG. 15 is an aerial view of a complete Patients'-Turn-A-kit.

FIG. 16 is a metal, plastic clamps, or restraint strap which is affixed to the bed railings.

FIG. 17 is a strap/belt made of industrial material with Velcro attached at both ends.

FIG. 18 is the vinyl center pad is used to protect and cushion skin that comes into contact with the device.

FIG. 19 is a disposable pillow casing for the center pad (FIG. 1) of the device.

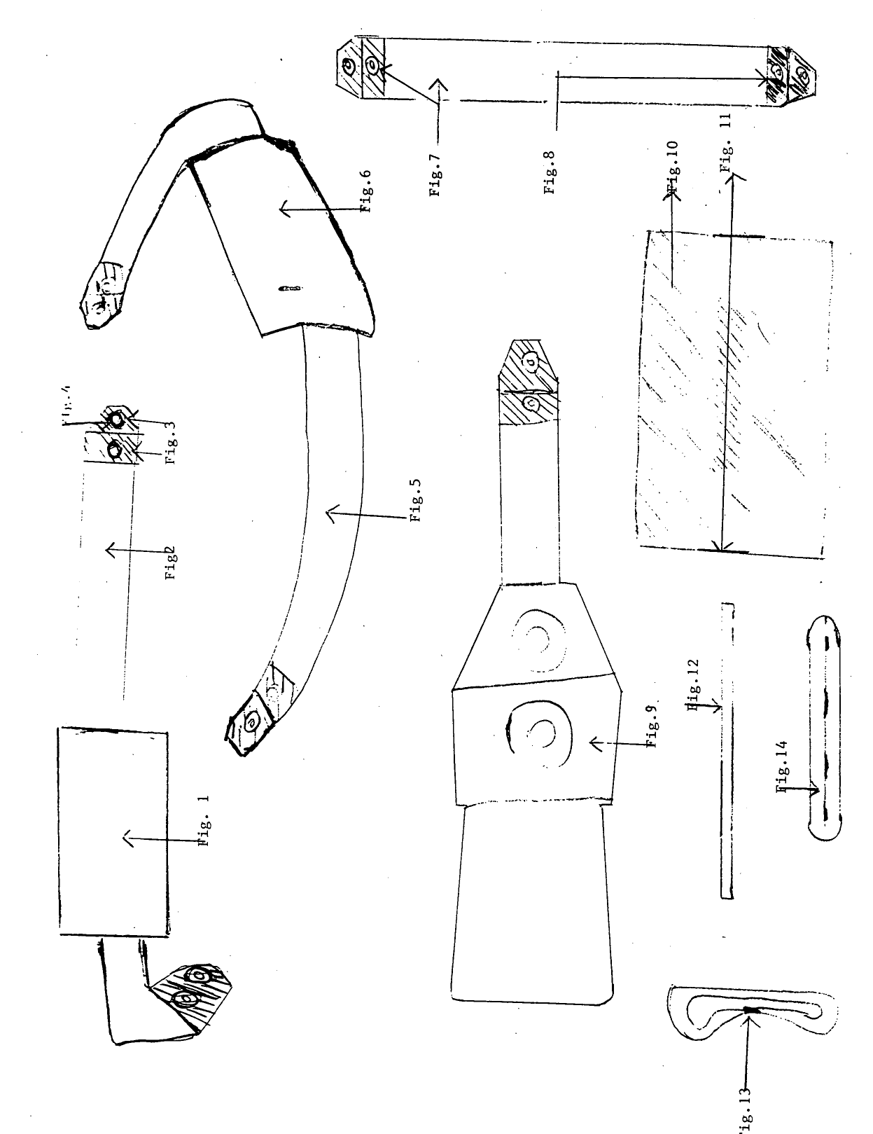

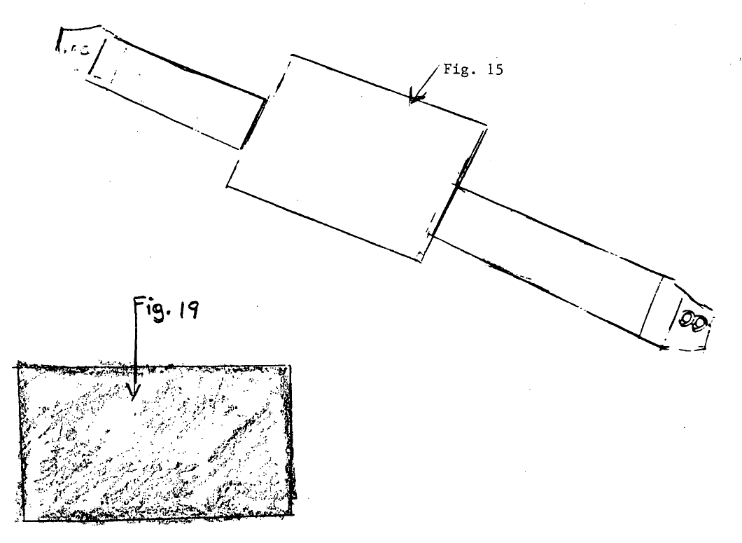

### DESCRIPTION OF THE PREFERRED EMBODIMENTS

As shown FIG. 15, the first preferred embodiment of the Patients Turn-A-Kit consist of (FIG. 1) a padded nylon center with slits on either side through which a (FIG. 2) strap/belt is threaded. At the end of each strap is (FIG. 3) a pair grommets through which can be threaded metal or plastics clamps, or restraining straps to affix the device to the bed railing. Velcro (FIG. 8) adheres to the end strap/belt at both ends and to the center padded nylon cushion. This holds the cushioning center in place once the padded nylon cushion has been properly placed FIG. 18 against the patient's skin. FIG. 8 is the center of the foam padding has additional nylon on which strips of Velcro are attached. FIG. 7 is the belt is made of an industrial fabric mesh material and can be adjusted from either side to increase or decrease the length of the strap/belt by simply pulling the strap/belt from either left or right. This will allow for the strap/belt to be extended up to a length of preferred length of 6 feet and decreased to the preferred length of 4 feet 8 inches depending on the size of the patient. FIG. 3 the grommets on each end are the preferred length of one inch in diameter with a hole of a preferred ⅜ of an inch. The padded nylon cushion center is manufactured as two (2) separate pads which are then folded to make a single pad. The nylon material is first wrapped around a piece of medical grade polyester fiber filled wrapped and sewn as a package. The padded cushion (FIG. 1) has slits on each end in which the preferred is 4 inches wide for the insertion of the strap/belt (FIG. 2). At the edge of each slit (FIG. 1) is a 1 inch wide strip of Velcro which adheres after insertion of strap/belt (FIG. 2) to aid in holding the padded cushion

(FIG. 1) in position. The padded cushion (FIG. 1) is preferred to be one foot wide and 15 inches long.

The strap/belt (FIG. 2) consists of an industrial material and is threaded through the two slits within the padded cushion. Each end of the strap will be tapered from a 4 inch width to the preferred of 3 inches in width and contains two brass grommets on each end that are preferred to be three inches apart. The grommets brass and are affixed to the strap/belt. The Velcro (FIG. 3) is located at each end of the slits on the padded nylon cushion this will aid with holding the position of the strap/belt.

### (FIG. 17) shows the Patient Turn-A-Kit on the patient while laying on his/her side and the device is attached to the bed rails.

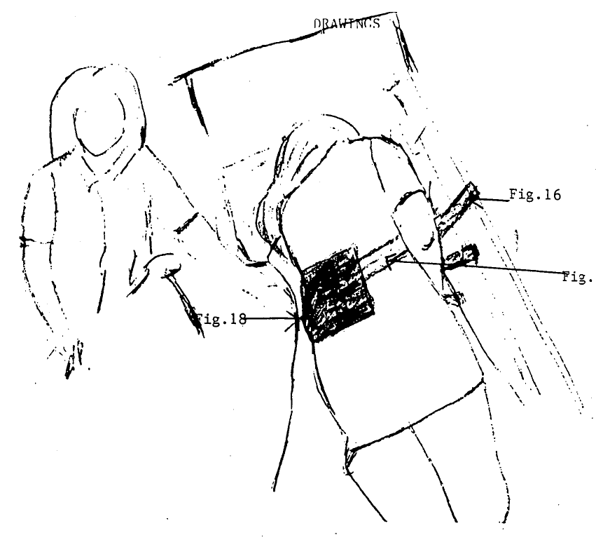

The device (FIG. 18) is placed underneath the patient until the pad is located that area of the patients' waist area where it will cause the least discomfort. The strap/belt extending from the padded cushion directly under the patient will be properly affixed to the lower bed rail. The top strap/belt comes over the patients' waist area and then affixes to the top rail. When turning the patient to the opposite side, the care provider merely unclips the metal, plastic clamps, or restraining strap from the bottom bed rail (FIG. 16) and placing the metal, plastic clamps or restraining straps to the opposite side of the bed rails and repeating the process. Once the device is placed underneath the waist area of the patient FIGS. 16, 17, and 18) there will be no further need to remove the device until the care providers' work is complete. Once completed, the disposable pillow case is removed from the center padding and discarded and replaced with a clean one.

What it claims are:

1. This invention can be used in nursing homes, hospitals, private homes and other facilities to turn their patients with safety and ease thereby reducing the effects of bed sores on non-ambulatory patients that have to be turned regularly. It will also make it easier to bath the patient and change the bedding. The device is user friendly and it is not bulky.
